(19) United States
(12) Patent Application Publication
(10) Pub. No.: US 2025/0195250 A1
(43) Pub. Date: Jun. 19, 2025

(54) APPARATUS AND METHOD FOR CONTROLLING LOWER-LIMB WEARING ROBOT BASED ON AUGMENTED REALITY AND BRAIN-COMPUTER INTERFACE

(71) Applicants: Hyundai Motor Company, Seoul (KR); Kia Corporation, Seoul (KR); IUCF-HYU (Industry-University Cooperation Foundation Hanyang University), Seoul (KR)

(72) Inventors: Seungkyu NAM, Seoul (KR); Kukhyun Ahn, Whasung-Si (KR); Taejun Lee, Whasung-Si (KR); Hoseung Cha, Whasung-Si (KR); Chang-Hwan Im, Whasung-Si (KR); Jinuk Kwon, Whasung-Si (KR); Hyo Jeong Han, Whasung-Si (KR)

(73) Assignees: Hyundai Motor Company, Seoul (KR); Kia Corporation, Seoul (KR); IUCF-HUY (Industry-University Cooperation Foundation Hanyang University), Seoul (KR)

(21) Appl. No.: 18/744,227

(22) Filed: Jun. 14, 2024

(30) Foreign Application Priority Data
Dec. 14, 2023 (KR) 10-2023-0181849

(51) Int. Cl. A61F 4/00; A61H 1/02; A61H 3/02; A61H 5/00; B25J 9/00; G06F 3/01; G16H 40/63

(52) U.S. Cl. CPC A61F 4/00; A61H 1/0237; A61H 3/02; A61H 5/00; B25J 9/0006; G06F 3/015; G16H 40/63; A61H 2201/1604; A61H 2201/165; A61H 2201/5043; A61H 2230/105

(57) ABSTRACT

An apparatus for controlling a lower-limb wearable robot based on augmented reality and brain-computer interface may include an augmented reality glasses configured to display a visual stimulus and status information of the lower-limb wearable robot through the augmented reality, a biological signal measurement module configured for measuring a biological signal of a user generated based on the visual stimulus and the status information of the lower-limb wearable robot, a control module configured to detect a control intention of the user based on the biological signal and generate the visual stimulus and the status information of the lower-limb wearable robot, and the lower-limb wearable robot configured to operate according to the control intention of the user.

### CROSS-REFERENCE TO RELATED APPLICATION

The present application claims priority to Korean Patent Application No. 10-2023-0181849 filed on Dec. 14, 2023, the entire contents of which is incorporated herein for all purposes by this reference.

### BACKGROUND OF THE PRESENT DISCLOSURE

### Field of the Present Disclosure

The present disclosure relates to an apparatus and method for controlling a lower-limb wearable robot based on augmented reality and brain-computer interface. More particularly, the present disclosure relates to an apparatus and method for controlling a lower-limb wearable robot based on augmented reality and brain-computer interface capable of controlling a brain-computer interface-based lower-limb wearable robot in a moving environment by use of augmented reality.

### Description of Related Art

Lower-limb wearable robots may be controlled by use of physical buttons such as crutches. When a paraplegic patient walks after wearing robot, there is a high dependence on leaning on the arms, e.g., by supporting strong weight by crutches at both sides, resulting in difficulty and in convenience in button control. To compensate for this and improve walking concentration, there is a need to develop a biological signal-based interface that controls the robot by reading the user's intention.

In the case of the existing brain-computer interface (BCI)-based lower-limb wearable robot control system using steady-state visual evoked potential (SSVEP), a light-emitting diode (LED) or LCD monitor may be used as an apparatus for presenting a visual stimulus for evoking the SSVEP. The SSVEP apparatus for presenting a visual stimulus may present difficulties in using in a moving environment. Furthermore, in the case of LED stimulation, there is a problem that the user's visual fatigue increases when exposed for a long time due to the influence of light.

The information included in this Background of the present disclosure is only for enhancement of understanding of the general background of the present disclosure and may not be taken as an acknowledgement or any form of suggestion that this information forms the prior art already known to a person skilled in the art.

### BRIEF SUMMARY

Various aspects of the present disclosure are directed to providing an apparatus and method for controlling a lower-limb wearable robot based on augmented reality and brain-computer interface, applied with a technique for presenting a visual stimulus using an augmented reality (AR) glass, for practical and convenient control of a lower-limb wearable robot in a moving environment.

The present disclosure attempts to provide an apparatus and method for controlling a lower-limb wearable robot based on augmented reality and brain-computer interface that provides an augmented reality-based interface to strengthen the interaction between the user and the robot, and provides an intuitive method of using the crutch to the user through a crutch position guide.

The present disclosure attempts to provide an apparatus and method for controlling a lower-limb wearable robot based on augmented reality and brain-computer interface, which may be used by anyone on their own without learning, by a dry electrode-based system.

An apparatus for controlling a lower-limb wearable robot based on augmented reality and brain-computer interface may include an augmented reality glasses configured to display a visual stimulus and status information of the lower-limb wearable robot through the augmented reality, a biological signal measurement module configured for measuring a biological signal of a user generated based on the visual stimulus and the status information of the lower-limb wearable robot, a control module configured to detect a control intention of the user based on the biological signal and generate the visual stimulus and the status information of the lower-limb wearable robot, and the lower-limb wearable robot configured to operate according to the control intention of the user.

The biological signal measurement module may include a first biological signal measurement module configured for measuring a first biological signal including electroencephalogram information of the user gazing at the visual stimulus, and a second biological signal measurement module configured for measuring a second biological signal generated by the user based on the status information of the lower-limb wearable robot.

The control module may include a brain-computer interface (BCI) calculating unit configured to classify a first control intention of the user based on the first biological signal and determine a second control intention of the user based on the second biological signal, and an augmented reality (AR) calculating unit configured to generate the visual stimulus and the status information of the lower-limb wearable robot based on the second control intention.

The control module may be configured to divide the second biological signal into a first signal and a second signal, switch the state of the lower-limb wearable robot from a stand state to a sit state based on the first signal when a state of the lower-limb wearable robot is a pause state, and switch the state of the lower-limb wearable robot from the sit state to the stand state based on the second signal.

When a state of the lower-limb wearable robot is a stand state in a pause state, the control module may be configured to switch the state of the lower-limb wearable robot to an operation state based on the second biological signal.

When the state of the lower-limb wearable robot is the operation state, the control module may be configured to determine an operation mode based on the first biological signal.

When the lower-limb wearable robot is in the operation mode, the control module may be configured to control an operation corresponding to the determined operation mode of the lower-limb wearable robot based on the second biological signal.

The control module may be configured to generate a crutch position guide configured to indicate a position to which a crutch of the user is to move, to correspond to the controlled operation of the lower-limb wearable robot in real time.

The augmented reality glasses may be configured to display the crutch position guide at a second location spaced from a first location where the visual stimulus and the status information of the lower-limb wearable robot are displayed and closer to the ground than the first location.

The control module may be configured to generate a plurality of visual stimuli that are based on steady-state visual evoked potential (SSVEP) and flickering with different frequencies.

A method for controlling a lower-limb wearable robot based on augmented reality and brain-computer interface may include displaying visual stimulus and status information of the lower-limb wearable robot to a user through an augmented reality glasses, measuring a biological signal of the user generated based on the visual stimulus and the status information of the lower-limb wearable robot, generating the visual stimulus and the status information of the lower-limb wearable robot based on the biological signal, detecting a control intention of the user based on the biological signal, and controlling an operation of the lower-limb wearable robot according to the control intention of the user.

The measuring the user's biological signal may include measuring a first biological signal including electroencephalogram information of the user gazing at the visual stimulus, and measuring a second biological signal generated by the user based on the status information of the lower-limb wearable robot.

The detecting the control intention of the user may include classifying a first control intention of the user based on the first biological signal, and determine a second control intention of the user based on the second biological signal, and the generating the status information of the lower-limb wearable robot may include generating the visual stimulus and the status information of the lower-limb wearable robot based on the second control intention.

The generating the status information of the lower-limb wearable robot may include dividing the second biological signal into a first signal and a second signal, switching the state of the lower-limb wearable robot from a stand state to a sit state based on the first signal, when a state of the lower-limb wearable robot is a pause state, and switching the state of the lower-limb wearable robot from the sit state to the stand state based on the second signal.

The generating the status information of the lower-limb wearable robot may include, when a state of the lower-limb wearable robot is a stand state in a pause state, switching the state of the lower-limb wearable robot to an operation state based on the second biological signal.

The detecting the control intention of the user may include, when the state of the lower-limb wearable robot is the operation state, determining an operation mode based on the first biological signal.

The controlling the operation of the lower-limb wearable robot may further include, when the lower-limb wearable robot is in the operation mode, controlling an operation corresponding to the determined operation mode of the lower-limb wearable robot based on the second biological signal.

A method for controlling a lower-limb wearable robot based on augmented reality and brain-computer interface may further include generating a crutch position guide configured to indicate a position to which a crutch of the user is to move, to correspond to the controlled operation of the lower-limb wearable robot in real time.

A method for controlling a lower-limb wearable robot based on augmented reality and brain-computer interface may further include display the crutch position guide at a second location spaced from a first location where the visual stimulus and the status information of the lower-limb wearable robot are displayed and closer to the ground than the first location through the augmented reality glasses.

The generating the visual stimulus and the status information of the lower-limb wearable robot may include generating a plurality of visual stimuli that are based on steady-state visual evoked potential (SSVEP) and flickering with different frequencies.

According to an apparatus and method for controlling a lower-limb wearable robot based on augmented reality and brain-computer interface according to an exemplary embodiment of the present disclosure, to control the lower-limb wearable robot, a technique for presenting a visual stimulus using an augmented reality (AR) glass is applied, being practical and convenient in the moving environment.

According to an apparatus and method for controlling a lower-limb wearable robot based on augmented reality and brain-computer interface according to an exemplary embodiment of the present disclosure, the interaction between the user and the robot may be strengthened by the augmented reality-based interface, and an intuitive method of using the crutch may be provided to the user through a crutch position guide based on the augmented reality.

According to an apparatus and method for controlling a lower-limb wearable robot based on augmented reality and brain-computer interface according to an exemplary embodiment of the present disclosure, a dry electrode-based system may be used to provide quick wearing and use, thereby being practical.

According to an apparatus and method for controlling a lower-limb wearable robot based on augmented reality and brain-computer interface according to an exemplary embodiment of the present disclosure, learning in advance is not required, and multiple biological signal may be utilized so that easy, quick, and accurate control of the robot may be enabled.

The methods and apparatuses of the present disclosure have other features and advantages which will be apparent from or are set forth in more detail in the accompanying drawings, which are incorporated herein, and the following Detailed Description, which together serve to explain certain principles of the present disclosure.

### BRIEF DESCRIPTION OF THE DRAWINGS

FIG. 1 shows an apparatus for controlling a lower-limb wearable robot based on augmented reality and brain-computer interface according to an exemplary embodiment of the present disclosure.

FIG. 2 is a flowchart showing a method for controlling a lower-limb wearable robot based on augmented reality and brain-computer interface according to an exemplary embodiment of the present disclosure.

FIG. 3 shows an operation mechanism of an apparatus for controlling a lower-limb wearable robot based on augmented reality and brain-computer interface according to an exemplary embodiment of the present disclosure.

FIG. 4 and FIG. 5A, FIG. 5B, FIG. 5C and FIG. 5D are drawings showing display of states of a lower-limb wearable robot through an augmented reality glasses according to an exemplary embodiment of the present disclosure.

FIG. 6 is a drawing showing a crutch guide displayed through an augmented reality glasses according to an exemplary embodiment of the present disclosure.

FIG. 7A, FIG. 7B, and FIG. 7C shows compensation of location of a visual stimulus displayed through an augmented reality glasses according to an exemplary embodiment of the present disclosure.

It may be understood that the appended drawings are not necessarily to scale, presenting a somewhat simplified representation of various features illustrative of the basic principles of the present disclosure. The specific design features of the present disclosure as included herein, including, for example, specific dimensions, orientations, locations, and shapes will be determined in part by the particularly intended application and use environment.

In the figures, reference numbers refer to the same or equivalent parts of the present disclosure throughout the several figures of the drawing.

### DETAILED DESCRIPTION

Reference will now be made in detail to various embodiments of the present disclosure(s), examples of which are illustrated in the accompanying drawings and described below. While the present disclosure(s) will be described in conjunction with exemplary embodiments of the present disclosure, it will be understood that the present description is not intended to limit the present disclosure(s) to those exemplary embodiments of the present disclosure. On the other hand, the present disclosure(s) is/are intended to cover not only the exemplary embodiments of the present disclosure, but also various alternatives, modifications, equivalents and other embodiments, which may be included within the spirit and scope of the present disclosure as defined by the appended claims.

An exemplary embodiment of the present disclosure will be described more fully hereinafter with reference to the accompanying drawings so that a person skill in the art may easily implement the embodiment. As those skilled in the art would realize, the described embodiments may be modified in various different ways, all without departing from the spirit or scope of the present disclosure. To clarify the present disclosure, parts that are not related to the description will be omitted, and the same elements or equivalents are referred to with the same reference numerals throughout the specification.

Furthermore, unless explicitly described to the contrary, the word “comprise” and variations such as “comprises” or “comprising” will be understood to imply the inclusion of stated elements but not the exclusion of any other elements. Terms including an ordinary number, such as first and second, are used for describing various constituent elements, but the constituent elements are not limited by the terms. The terms are only used to differentiate one component from other components.

Furthermore, the terms “unit”, “part” or “portion”, “-er”, and “module” in the specification refer to a unit that processes at least one function or operation, which may be implemented by hardware, software, or a combination of hardware and software.

Hereinafter, various exemplary embodiments of the present disclosure will be described with reference to the drawings.

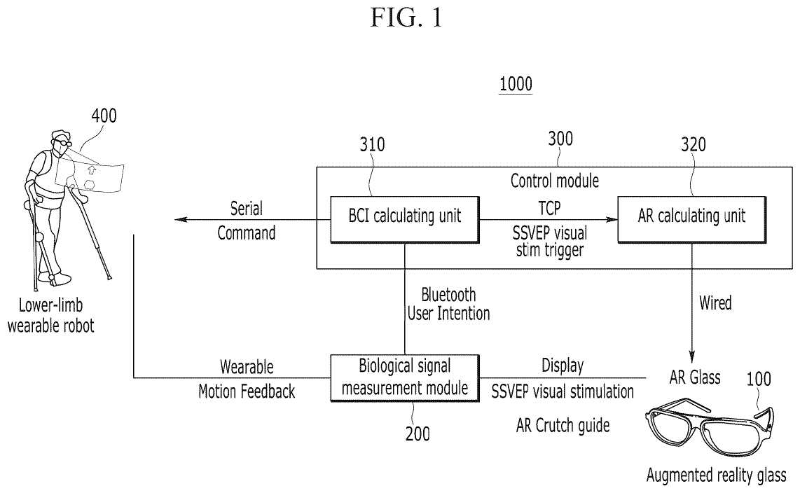

FIG. 1 shows an apparatus for controlling a lower-limb wearable robot based on augmented reality and brain-computer interface according to an exemplary embodiment of the present disclosure.

Referring to FIG. 1, an apparatus 1000 for controlling a lower-limb wearable robot based on augmented reality and brain-computer interface may include an augmented reality glasses 100, a biological signal measurement module 200, a control module 300, and a lower-limb wearable robot 400.

In an exemplary embodiment of the present disclosure, the apparatus 1000 for controlling the lower-limb wearable robot based on augmented reality and brain-computer interface may be provided in the lower-limb wearable robot 400. That is, the augmented reality glasses 100, the biological signal measurement module 200, and the control module 300 may be provided in the lower-limb wearable robot 400.

The augmented reality glasses 100 may display a visual stimulus and status information of the lower-limb wearable robot through the augmented reality (AR). The augmented reality glasses 100 may display the visual stimulus and state or operation information of the lower-limb wearable robot generated through the control module 300.

The augmented reality glasses 100 may display a crutch position guide at a second location spaced from a first location where the visual stimulus and the status information of the lower-limb wearable robot are displayed and closer to the ground than the first location.

For example, the augmented reality glasses 100 may display a status window showing status information of the lower-limb wearable robot 400 on the front of AR glasses in conjunction with the user's head movement.

For example, the augmented reality glasses 100 may fixedly display steady-state visual evoked potential (SSVEP) evoking visual stimulus and the crutch position guide in a floor regardless of the user's head movement.

The augmented reality glasses 100 may be worn by the user together with the lower-limb wearable robot and provide control convenience to the user gazing at the lower-limb wearable robot status information and the visual stimulus displayed to the user.

The biological signal measurement module 200 may measure the user's biological signal generated based on the visual stimulus and the status information of the lower-limb wearable robot.

The biological signal measurement module 200 may be a small multi-biological signal dedicated measurement equipment configured for reducing the number of unnecessary channels, and may be implemented in a band-typed wearing manner.

The biological signal measurement module 200 utilizes dry electrodes to reduce the consumption time due to the injection of gel to increase conductivity between the scalp skin and the electrodes, and improves wearing convenience by eliminating discomfort after using the gel.

The biological signal measurement module 200 may be fixed to the augmented reality glasses 100. In other words, the biological signal measurement module 200 may be fixed to the augmented reality glasses 100 by a ring because the augmented reality glasses 100 may fall as the user looks downward when the lower-limb wearable robot 400 walks.

The biological signal measurement module 200 may be a device configured for wireless communication based on Bluetooth, may improve convenience of wearing and moving, and may be implemented as a wired device depending on need or environment.

The biological signal measurement module 200 may include a first biological signal measurement module and/or a second biological signal measurement module.

The first biological signal measurement module may measure a first biological signal including electroencephalogram information of the user gazing at the visual stimulus. For example, the first biological signal may be an electroencephalography (EEG) signal, and the first biological signal measurement module may be an EEG signal measurement equipment including an EEG electrode worn near to the user's occipital lobe.

The first biological signal measurement module may use eight electrodes near to the occipital lobe as the EEG electrode, or the number of electrodes may be increased or decreased as needed.

The first biological signal measurement module may measure electroencephalogram information due to a neural response generated by the SSVEP evoking visual stimulus. The SSVEP is a neural response that occurs near to the occipital lobe due to a visual stimulus including a specific frequency. The first biological signal measurement module may an SSVEP-dedicated measurement equipment.

The second biological signal measurement module may measure a second biological signal generated by the user based on the status information of the lower-limb wearable robot. For example, the second biological signal may be electrooculography (EOG) signal, and the second biological signal measurement module may be an EOG signal measurement equipment worn near user's frontal lobe.

The second biological signal measurement module may include an EOG electrode which is fixed to and worn through a band near to the user's eyebrows. The EOG electrode may use two electrodes near to the forehead, and may be detachable from the main body, to be removed as needed.

The EEG electrode and the EOG electrode may use a dry electrode or wet electrode.

Electroencephalography (EEG) and/or electrooculography (EOG) signals of the user measured by the biological signal measurement module 200 may be transferred to the control module 300 including a brain-computer interface (BCI) system for analysis for controlling the lower-limb wearable robot. For example, the biological signal measurement module 200 may be wirelessly connected to the control module 200 and transmit the user's biological signals in real time.

The control module 300 may detect control intention of the user based on the biological signal, and may be configured to generate the visual stimulus and the status information of the lower-limb wearable robot. The control module 300 may be connected to a separate Bluetooth wireless controller for user safety, and may also manually control the operation of the lower-limb wearable robot using the wireless controller.

The control module 300 may be configured to generate a plurality of visual stimuli that are based on steady-state visual evoked potential (SSVEP) and flickering with different frequencies.

The control module 300 may include a brain-computer interface (BCI) calculating unit 310 and an augmented reality (AR) calculating unit 320.

The BCI calculating unit 310 may classify a first control intention of the user based on the first biological signal including the EEG signal, and may be configured to determine a second control intention of the user based on the second biological signal including the EOG signal. Here, the first control intention may be configured to determine an operation mode of the lower-limb wearable robot 400. The second control intention may be configured to determine a pause state or an operation state of the lower-limb wearable robot 400.

The BCI calculating unit 310 may receive EEG and/or EOG data based on the plurality of visual stimuli and may classify or detect the control intention of the user.

The BCI calculating unit 310 may transfer operation or status information of the lower-limb wearable robot generated based on the control intention of the user to the AR calculating unit 320 and the lower-limb wearable robot 400. The BCI calculation unit 310 may perform TCP wireless communication with the AR calculation unit 320, and may transfer SSVEP visual stimulation information and status information of the lower-limb wearable robot.

The AR calculating unit 320 may be configured to generate the visual stimulus and the status information of the lower-limb wearable robot based on the user's second control intention. The AR calculating unit 320 may present the visual stimulus based on the second control intention identified based on the EOG signal or generate status information of the lower-limb wearable robot.

The AR calculating unit 320 may be configured to generate the operation mode of the lower-limb wearable robot with the first control intention of the user gazing at the visual stimulus generated according to the second control intention.

The AR calculating unit 320 may be configured to generate SSVEP visual stimulation, status information of the lower-limb wearable robot, and the crutch position guide according to the recognition result with respect to the control intention of the user determined according to the BCI calculating unit 310, and may output them through the augmented reality glasses 100.

The AR calculation unit 320 may be integrated with the BCI calculation unit 310 and operated integrally in the control module 300.

In an exemplary embodiment of the present disclosure, the control module 300 may divide the second biological signal including the EOG signal into a first signal and a second signal, and when the state of the lower-limb wearable robot is the pause state, may switch the state of the lower-limb wearable robot from a stand state to a sit state based on the first signal, and may switch the state of the lower-limb wearable robot from the sit state to the stand state based on the second signal. Here, the first signal may be one of a left winking or a right winking, and the second signal may be the other one thereof.

When the state of the lower-limb wearable robot is the stand state in the pause state, the control module 300 may switch the state of the lower-limb wearable robot to the operation state based on the second biological signal.

When the state of the lower-limb wearable robot is the operation state, the control module 300 may be configured to determine the operation mode based on the first biological signal. For example, the operation mode may include a step mode, a walk mode, and a turn mode.

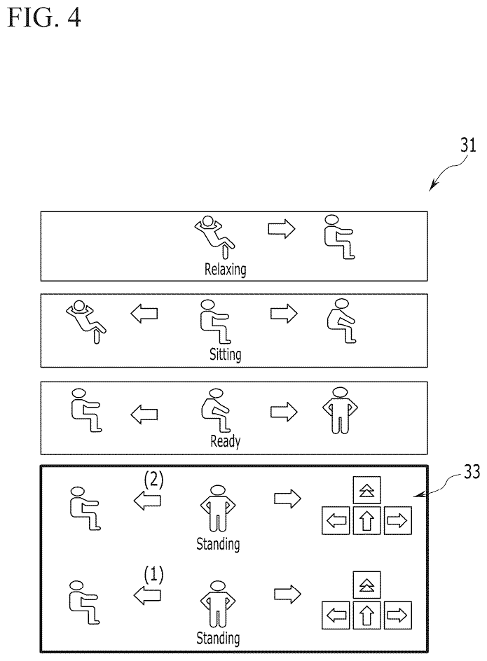

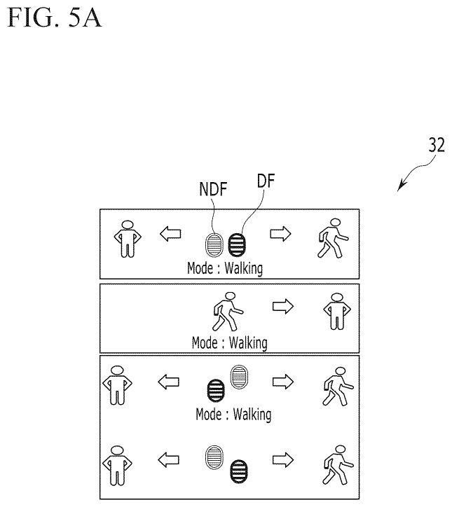

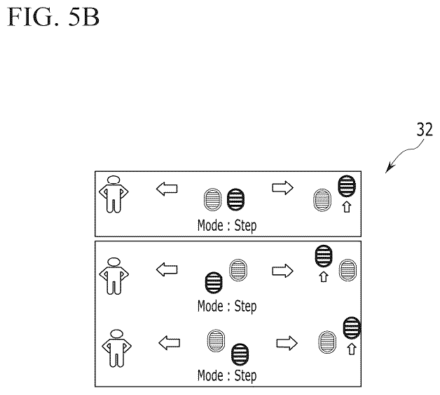

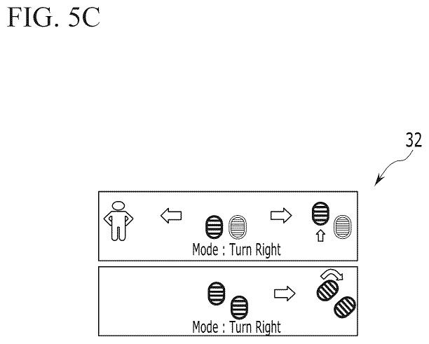

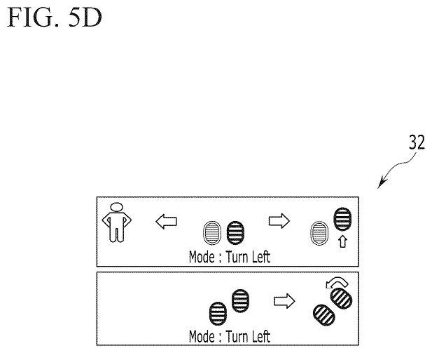

When the lower-limb wearable robot is in the operation mode, the control module 300 may be configured for controlling an operation corresponding to the determined operation mode of the lower-limb wearable robot based on the second biological signal. This will be described in detail with reference to FIG. 4 and FIG. 5A, FIG. 5B, FIG. 5C and FIG. 5D.

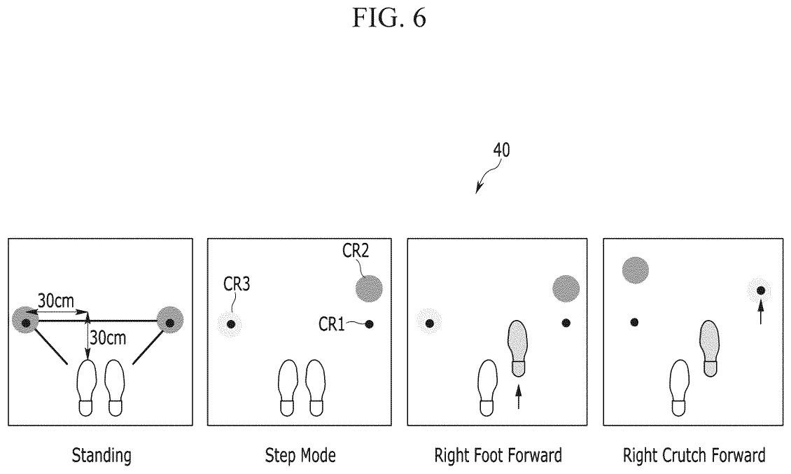

The control module 300 may be configured to generate the crutch position guide that indicates the position to which the user's crutch needs to move, to correspond to controlled operation of the lower-limb wearable robot in real time. This will be described in detail with reference to FIG. 6.

The lower-limb wearable robot 400 may operate according to the control intention of the user.

The lower-limb wearable robot 400 is configured to perform wired serial communication with the control module 300, and may perform the operation according to the recognition result of the user's intention of the control module 300.

The user may receive an operation feedback from the lower-limb wearable robot 400 operating according to the intention result. By the status information of the lower-limb wearable robot displayed on the augmented reality glasses 100, the user may continuously know information on the current state of the robot and an available state configured for next motion.

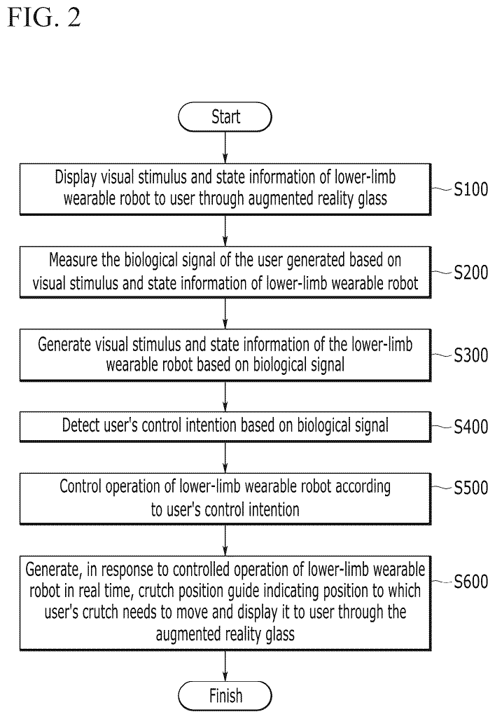

FIG. 2 is a flowchart showing a method for controlling the lower-limb wearable robot based on augmented reality and brain-computer interface according to an exemplary embodiment of the present disclosure. A method for controlling the lower-limb wearable robot based on augmented reality and brain-computer interface may be performed through the apparatus 1000 for controlling the lower-limb wearable robot based on augmented reality and brain-computer interface.

In FIG. 2, at step S100, the apparatus 1000 for controlling the lower-limb wearable robot based on augmented reality and brain-computer interface may display the visual stimulus and the status information of the lower-limb wearable robot to the user through the augmented reality glasses.

In an exemplary embodiment of the present disclosure, the apparatus 1000 for controlling the lower-limb wearable robot based on augmented reality and brain-computer interface may display the crutch position guide at the second location which is spaced from the first location where the visual stimulus and the status information of the lower-limb wearable robot are displayed and closer to the ground than the first location through the augmented reality glasses.

At step S200, the apparatus 1000 for controlling the lower-limb wearable robot based on augmented reality and brain-computer interface may measure the user's biological signal generated based on the visual stimulus and the status information of the lower-limb wearable robot.

The apparatus 1000 for controlling the lower-limb wearable robot based on augmented reality and brain-computer interface may measure the first biological signal including electroencephalogram information of the user gazing at the visual stimulus, and may measure the second biological signal generated by the user based on the status information of the lower-limb wearable robot. The first biological signal may be the EEG signal including the user's electroencephalogram information, and the second biological signal may be the EOG signal from which the user's eye movement may be known.

At step S300, the apparatus 1000 for controlling the lower-limb wearable robot based on augmented reality and brain-computer interface may be configured to generate the visual stimulus and the status information of the lower-limb wearable robot based on the biological signal.

The apparatus 1000 for controlling the lower-limb wearable robot based on augmented reality and brain-computer interface may be configured to generate the plurality of visual stimuli that are based on steady-state visual evoked potential (SSVEP) and flickering with different frequencies.

At step S400, the apparatus 1000 for controlling the lower-limb wearable robot based on augmented reality and brain-computer interface may detect the control intention of the user based on the biological signal.

The apparatus 1000 for controlling the lower-limb wearable robot based on augmented reality and brain-computer interface may classify the first control intention of the user related to the operation mode of the lower-limb wearable robot based on the first biological signal, and may be configured to determine the second control intention of the user related to the switching of the pause state or the operation state of the lower-limb wearable robot or the operation of the operation state based on the second biological signal.

The apparatus 1000 for controlling the lower-limb wearable robot based on augmented reality and brain-computer interface may divide the second biological signal into the first signal with respect to one of the left winking or the right winking and the second signal with respect to the other thereof. The apparatus 1000 for controlling the lower-limb wearable robot based on augmented reality and brain-computer interface may differently control state switching of the lower-limb wearable robot depending on the first signal or the second signal each.

When the state of the lower-limb wearable robot is the operation state, the apparatus 1000 for controlling the lower-limb wearable robot based on augmented reality and brain-computer interface may be configured to determine the operation mode based on the first biological signal.

The apparatus 1000 for controlling the lower-limb wearable robot based on augmented reality and brain-computer interface may be configured to generate the visual stimulus and the status information of the lower-limb wearable robot based on the user's second control intention.

At step S500, the apparatus 1000 for controlling the lower-limb wearable robot based on augmented reality and brain-computer interface may be configured for controlling the operation of the lower-limb wearable robot according to the control intention of the user.

When the state of the lower-limb wearable robot is the pause state, the apparatus 1000 for controlling the lower-limb wearable robot based on augmented reality and brain-computer interface may switch state of the lower-limb wearable robot from the stand state to the sit state based on the first signal.

The apparatus 1000 for controlling the lower-limb wearable robot based on augmented reality and brain-computer interface may switch state of the lower-limb wearable robot from the sit state to the stand state based on the second signal.

When the state of the lower-limb wearable robot is the stand state in the pause state, the apparatus 1000 for controlling the lower-limb wearable robot based on augmented reality and brain-computer interface may switch the state of the lower-limb wearable robot to the operation state based on the second biological signal.

When the lower-limb wearable robot is in the operation mode, the apparatus 1000 for controlling the lower-limb wearable robot based on augmented reality and brain-computer interface may be configured for controlling the operation corresponding to the operation mode of the lower-limb wearable robot based on the second biological signal.

For example, the apparatus 1000 for controlling the lower-limb wearable robot based on augmented reality and brain-computer interface may perform the step operation of the lower-limb wearable robot, step by step, whenever the right winking of the user is performed in the step mode.

At step S600, the apparatus 1000 for controlling the lower-limb wearable robot based on augmented reality and brain-computer interface may be configured to generate the crutch position guide indicating the position to which the user's crutch needs to move and display it to the user through the augmented reality glasses, to correspond to controlled operation of the lower-limb wearable robot in real time.

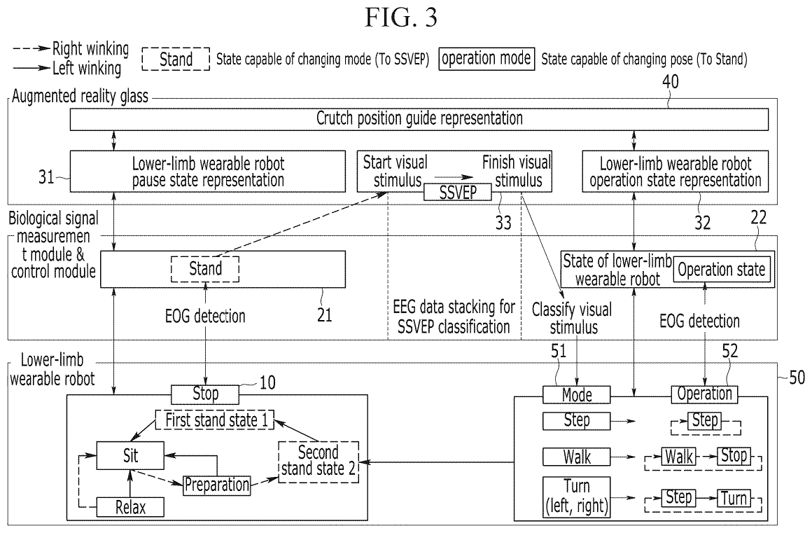

FIG. 3 shows an operation mechanism of an apparatus for controlling the lower-limb wearable robot based on augmented reality and brain-computer interface according to an exemplary embodiment of the present disclosure. Hereinafter, description will be made with reference to FIG. 1 and FIG. 2.

The state of the lower-limb wearable robot 400 may be largely divided into the pause state 10 and the operation (or movement) state 50.

The pause state 10 may be a basic posture, and may include a stand state, a sit state, a ready state, and a relax state.

The operation state 50 may include the operation mode 51. The operation mode 51 may include a step mode for walking step by step, a walk mode for continuously walking, and a turn mode including a right turn and a left turn.

The EOG signal measured through the biological signal measurement module 200 may be basically generated through a wink operation of the user.

The control module 300 may apply two control methods by distinguishing between the right winking and the left winking. This may be used mainly for executing the operation of the lower-limb wearable robot. The control module 300 may apply the two control methods to the execution of the operation to minimize eye fatigue generated when frequently using the SSVEP, and may remove the problem of time required for intention recognition through the SSVEP to perform the robot operation control accurately and rapidly.

The right winking may perform a dotted arrow command, and the left winking may perform a solid arrow command.

There may be a possibility unintended false positive result in the case of the conversion to the sit state from the stand state due to the left winking detection, and the control module 300 may distinguishably perform and count a first stand state 1 and a second stand state 2, i.e., into two states, for safety reasons.

In the operation mode 51, the control module 300 may switch the state of the lower-limb wearable robot back to the stand state 21 by the left winking, and when performing the operation mode 51, may switch back to the stand state 21 only at the time of finishing each operation 52.

In the execution of the operation, when needed as in the case of the user who has difficulty in the wink operation, the control module 300 may replace the wink-based EOG signal with the EOG signal such as continuously blinking, left and right gazing, or frowning or an electromyography (EMG) signal such as clenching.

The control module 300 may use the SSVEP signal to select the operation mode 51 for moving according to the intention of the user. For example, the control module 300 may present the SSVEP evoking visual stimulus 33 by the right winking motion only when the lower-limb wearable robot 400 is in the stand state 21, and may select the operation mode 51 through classification of the visual stimulus through SSVEP recognition in the operation state 22.

In an exemplary embodiment of the present disclosure, the time for which the SSVEP visual stimulation is presented may be a fixed time window, and the same stimulus may be presented for a preset time. Alternatively, the adaptive time window technique allows recognition of SSVEP visual stimulation at the optimal time for each control trial. While the SSVEP visual stimulation is presented, the user's concentration of intention and performance may be improved through online visual feedback techniques.

The control module 300 may use non-learning algorithms such as a canonical correlation analysis (CCA) and a filter bank canonical correlation analysis (FBCCA) that do not require prior learning for convenience and usability of the lower-limb wearable robot control as the SSVEP recognition algorithm.

The control module 300 may use a learning-based algorithm such as a deep learning technique such as a task-related component analysis (TRCA), a convolutional neural network (CNN), a Long Short-Term Memory (LSTM) that require learning, as needed, as the SSVEP recognition algorithm.

The status information of the lower-limb wearable robot may be shared with the control module 300 and the augmented reality glasses 100, and the user may confirm in rear time the information on next available operation and current status of the lower-limb wearable robot displayed through the augmented reality in the augmented reality glasses 100.

The user may receive in real time the crutch position guide with respect to the location of the crutch through the augmented reality glasses 100 based on information on movement operation of the wear robot.

As an exemplary embodiment of the present disclosure, through the apparatus 1000 for controlling the lower-limb wearable robot based on augmented reality and brain-computer interface, the lower-limb wearable robot may operate back to the sit state after performing the step operation from the relax state, as follows.

The apparatus 1000 for controlling the lower-limb wearable robot based on augmented reality and brain-computer interface may display the relax state as the pause state representation 31 of the lower-limb wearable robot in an augmented reality screen gazed at by the user through the augmented reality glasses 100. Furthermore, when performing the right winking as the next available operation, the augmented reality glasses 100 may display information of which the state is changed to the sit state.

When the user performs the right winking motion according to the intention to stand up, the control module 300 may detect the right winking signal and switches the lower-limb wearable robot 400 to the sit state.

The status information of the robot may be transferred to the AR calculating unit 320 through the BCI calculating unit 310, and the AR calculating unit 320 may output a current status information to the augmented reality glasses 100.

The augmented reality glasses 100 display the sit state on the AR screen viewed by the user. At the same time, when performing the right winking as the next available operation, the augmented reality glasses 100 may switch to the ready state, and when performing the left winking, may display information of which the state is switched to the relax state.

When the user performs the right winking motion again, the control module 300 may switch the state of the lower-limb wearable robot 400 to ready state.

The apparatus 1000 for controlling the lower-limb wearable robot based on augmented reality and brain-computer interface may display the ready state on the augmented reality screen of the augmented reality glasses 100 viewed by the user, and may display information in which the state of the lower-limb wearable robot is changed to the stand state when performing the right winking as the next available operation, and to the sit state when performing the left winking.

When the user performs the right winking motion again, the control module 300 may detect a biological signal of the right winking, and switch the lower-limb wearable robot 400 to the stand state. At the instant time, the user may maintain balance by leaning on the crutch according to the crutch position guide representation 40 displayed at the floor.

The control module 300 may display the stand state on the screen of the augmented reality glasses 100 viewed by the user. At the same time, the control module 300 may display that the SSVEP evoking visual stimulus 33 is displayed when performing the right winking as the next available operation through the augmented reality glasses 100, and display the step 2 for switching to the sit state as state conversion information when performing the left winking perform.

When the user performs the right winking motion again, the control module 300 may detect the right winking motion, and may display the SSVEP evoking visual stimulus 33 on the augmented reality glasses 100.

When the SSVEP evoking visual stimulus 33 is presented to the user through the augmented reality glasses 100 for a preset time and an SSVEP electroencephalogram signal is generated as the user gazes at the stimulus of the desired movement operation, the visual stimulation is finished and the control module 300 may classify the control intention of the user through the SSVEP recognition algorithm.

When the control intention of the user is classified as the step mode, current status screen of the augmented reality glasses 100 viewed by the user may display the step mode. Furthermore, the augmented reality glasses 100 may display information on the foot to move one step when performing the right winking as the next available operation, and information of which the state is changed to the stand state in the operation mode when performing the left winking.

When the user performs the right winking, the lower-limb wearable robot 400 may move forward one step for each the right winking. At the instant time, the status window of the augmented reality glasses 100 for the operation state representation 32 of the lower-limb wearable robot may change according to a current foot location. The crutch position guide representation 40 may continuously display the location where the crutch needs to placed next.

When the user performs the left winking, the lower-limb wearable robot 400 may put its legs together and switches to the stand state, the status window of the augmented reality glasses 100 may change to match the current status, and the crutch position guide representation 40 may also show the stand position.

When the user performs the left winking in the stand state, in the status window of the augmented reality glasses 100, the second stand state 2 may be counted down to the first stand state 1, and when the user performs the left winking again, the lower-limb wearable robot 400 may switch to the sit state.

FIG. 4 and FIG. 5A, FIG. 5B, FIG. 5C and FIG. 5D are drawings showing display of states of the lower-limb wearable robot through an augmented reality glasses according to an exemplary embodiment of the present disclosure.

FIG. 4 shows the pause state representations 31 (refer to FIG. 3) of the lower-limb wearable robot displayed on the augmented reality glasses in the pause state of the lower-limb wearable robot. That is, the apparatus 1000 for controlling the lower-limb wearable robot based on augmented reality and brain-computer interface may show the current state of the robot worn by the user with icons through the augmented reality screen so that the user may confirm the status information of the lower-limb wearable robot.

The status window showing the status information of the lower-limb wearable robot may be displayed to move according to the user's head movement so that the user may always find it in the central upper end portion of the screen of the augmented reality glasses.

The pause state may include the stand state in the standing posture, the sit state in the seated posture, the ready state of the ready posture, the relax state of the relaxing posture.

In FIG. 4, the central icon may represent current status of the lower-limb wearable robot.

A right arrow may represent a next operation processed when the user performs the right winking. A left arrow may represent a next operation processed when the user performs the left winking.

When switching from the stand state to the sit state according to false positive result occurs, a safety problem may occur. Therefore, when switching the state of the lower-limb wearable robot from the stand state to the sit state, the user needs to perform the left winking twice, and the required count may be confirmed at the top portion of the left arrow.

For example, when the user performs the left winking once, the count 2 may be changed to 1, and when the user performs the left winking once more, the lower-limb wearable robot is switched to the sit state.

Regardless of the count, in the stand state, when the user performs the right winking, the SSVEP evoking visual stimulus 33 is displayed on the AR screen and flickers. The apparatus 1000 for controlling the lower-limb wearable robot based on augmented reality and brain-computer interface may classify the operation mode target by the user the SSVEP recognition algorithm based on the biological information of the user gazing at the visual stimulus. When the classification with respect to the control intention of the user based on the SSVEP evoking visual stimulus 33 is finished, the pause state is switched to the operation state of the operation mode according to the control intention.

For example, in the SSVEP evoking visual stimulus 33, the double arrow block may induce the walk mode, the central arrow block may induce the step mode, the right-side arrow block may induce the turn mode to the right, and the left-side arrow block may induce the turn mode to the left.

FIG. 5A, FIG. 5B, FIG. 5C and FIG. 5D show various operation state representations 32 (refer to FIG. 3) of the lower-limb wearable robot displayed on the augmented reality glasses in the operation state of the lower-limb wearable robot.

The operation mode may include the step mode for walking step by step, the walk mode for walking continuously, and the turn mode to the left and the turn mode to right for turning to the left and to the right, respectively.

FIG. 5A shows the operation state representation 32 of the wearable robot displayed on the augmented reality glasses in the walk mode. FIG. 5B shows the operation state representation 32 of the wearable robot displayed on the augmented reality glasses in the step mode. FIG. 5C shows the operation state representation 32 of the wearable robot displayed on the augmented reality glasses in the turn mode to the right. FIG. 5D shows the operation state representation 32 of the wearable robot displayed on the augmented reality glasses in the turn mode to the left.

The uppermost end block of each operation mode of FIG. 5A, FIG. 5B, FIG. 5C, and FIG. 5D represents a first status window appearing after selecting the operation mode. Central icons of respective blocks may represent current status of the lower-limb wearable robot, and a designated foot DF may the foot to be actually moved when the present movement operation is executed. The designated foot DF and a non-designated foot NDF may be displayed as icons distinguishable from each other.

The right arrow represents a next operation processed when the user performs the right winking, and the foot and shape to be actually moved is indicated as the designated foot DF.

In FIG. 5A, when the user performs the right winking once in the walk mode, the lower-limb wearable robot may continue walking until the right winking is performed subsequently.

In FIG. 5B, when the user performs the right winking once in the step mode, the lower-limb wearable robot may take a step with the designated foot DF. When the user performs the right winking again, the lower-limb wearable robot may take a step with the designated foot DF which is subsequently designated.

In FIG. 5C, when the user performs the right winking once in the turn mode to the right, the lower-limb wearable robot may take a step with the designated foot DF. When the user performs the right winking again, the lower-limb wearable robot may rotate to the right around an axis of both feet.

In FIG. 5D, when the user performs the right winking once in the turn mode to the left, the lower-limb wearable robot may take a step with the designated foot DF. When the user performs the right winking again, the lower-limb wearable robot may rotate to the left around an axis of both feet.

The left arrow may represent a next operation processed when the user performs the left winking. In each operation mode of FIG. 5A, FIG. 5B, FIG. 5C, and FIG. 5D, when the left winking is performed, the legs are brought together, and the lower-limb wearable robot is switched to the stand state of the pause state. from the stand state, the user may select the operation mode again through BCI based on the SSVEP evoking visual stimulus.

FIG. 6 is a drawing showing a crutch guide displayed through the augmented reality glasses according to an exemplary embodiment of the present disclosure. FIG. 6 shows the crutch position guide representation 40 (refer to FIG. 3) displayed on the augmented reality glasses in the operation state of the lower-limb wearable robot.

The apparatus 1000 for controlling the lower-limb wearable robot based on augmented reality and brain-computer interface may guide the location of the crutch to be moved by the user through the augmented reality, helping to enable safe and balanced walking through the lower-limb wearable robot.

FIG. 6 shows the crutch position guide displayed through the augmented reality glasses for each movement of a lower-limb wearable robot in each operation mode.

The crutch position CR1 that the user is currently holding may be indicated as a dot. A first guide CR2 indicating the position where the crutches needs to move may be indicated as a circle. After the crutches are positioned at the first guide CR2, the first guide CR2 is switched to a second guide CR3. The first guide CR2 and the second guide CR3 may be displayed distinctly from each other in various ways. For example, the first guide CR2 and the second guide CR3 may be displayed as circles with different brightness and darkness.

The crutch position guide may be displayed fixed to the floor surface regardless of the user's head movement in augmented reality.

In FIG. 6, in the standing state, the user prepares for walking by positioning the crutches according to the crutch position guide on the floor to maintain balance. Afterwards, after switching to the step mode, the second guide CR3 guides the position of the crutches that the user is currently holding, and the first guide CR2 guides the user to the next position of the crutches that the user should hold.

When the lower-limb wearable robot performs operation in the step mode by the apparatus 1000 for controlling the lower-limb wearable robot based on augmented reality and brain-computer interface, the lower-limb wearable robot moves one foot first, and the crutch position guide may display the first guide CR2 and the second guide CR3.

The user may simply position the crutch according to the first guide CR2. When the movement of one step is completed, the crutch position guide may guide the position of the crutches to be moved according to the next step through the first guide CR2.

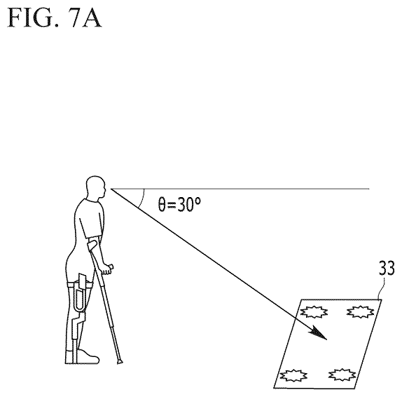

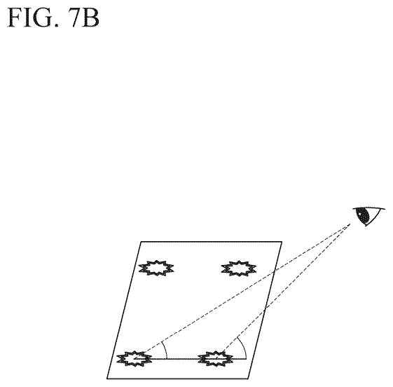

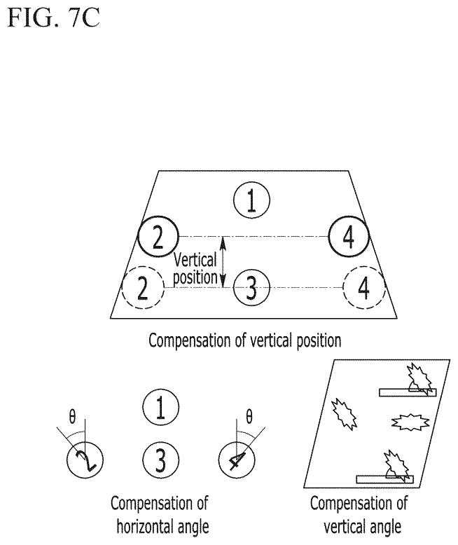

FIG. 7A, FIG. 7B, and FIG. 7C shows compensation of location of the SSVEP evoking visual stimulus 33 (refer to FIG. 3 and FIG. 4) displayed through the augmented reality glasses according to an exemplary embodiment of the present disclosure.

The SSVEP evoking visual stimulus 33 configured to control the operation mode may be formed in arrow shapes in four checkerboard patterns indicating the walk mode for continuously walking, the turn mode to the left, the step mode for walking step by step, and the turn mode to the right, respectively. Each visual stimulus may flicker at the particular frequency. For example, the frequency of the respective visual stimuli may be 6 Hz, 6.7 Hz, 7.5 Hz, 10 Hz. The checkerboard pattern may be changed to a basic flickering pattern or other forms.

In FIG. 7A and FIG. 7B, the SSVEP evoking visual stimulus 33 may be displayed in augmented reality by being fixed to the floor at a 30-degree angle of head bending of the user. Because excessive changes in the surrounding background may lead to deterioration of SSVEP performance, the apparatus 1000 for controlling the lower-limb wearable robot based on augmented reality and brain-computer interface may fix the SSVEP evoking visual stimulus 33 to the floor surface which may be maintained to have constant background.

Excessive head bending may not only cause neck discomfort, but may also cause noise signals caused by neck muscles, which may lead to deterioration of SSVEP performance, and therefore, the apparatus 1000 for controlling the lower-limb wearable robot based on augmented reality and brain-computer interface may set 30 degrees as the optimal bending degreed.

In the case of the continuous walking visual stimulus at the furthest top from the user, if it is set to the same size as the visual stimulus at the floor closest to the user, a problem occurs where the magnitude of the actual visible stimulus area varies due to distortion depending on the projection angle of the field of view, which causes deterioration of SSVEP performance.

In an exemplary embodiment of the present disclosure, the apparatus 1000 for controlling the lower-limb wearable robot based on augmented reality and brain-computer interface may perform horizontal position correction to make the upper and lower stimuli appear to be the same size.

In FIG. 7C, the positions of the visual stimuli 2 and 4 at the bottom may be corrected in the vertical direction so that they appear to be the same or similar in size to the visual stimuli 1 at the top.

Furthermore, due to the small projection angle of the visual stimulus at the top, causing problems of uncomfortable viewing and poor SSVEP performance, the apparatus 1000 for controlling the lower-limb wearable robot based on augmented reality and brain-computer interface may perform vertical angle and horizontal angle optimization correction of the SSVEP evoking visual stimulus.

For example, in the case of the visual stimuli 2 and 4 at the bottom portion, they may be corrected by a predetermined angle horizontally and vertically in consideration of the user's field of view.

Furthermore, the term related to a control device such as “controller”, “control apparatus”, “control unit”, “control device”, “control module”, or “server”, etc refers to a hardware device including a memory and a processor configured to execute one or more steps interpreted as an algorithm structure. The memory stores algorithm steps, and the processor executes the algorithm steps to perform one or more processes of a method in accordance with various exemplary embodiments of the present disclosure. The control device according to exemplary embodiments of the present disclosure may be implemented through a nonvolatile memory configured to store algorithms for controlling operation of various components of a vehicle or data about software commands for executing the algorithms, and a processor configured to perform operation to be described above using the data stored in the memory. The memory and the processor may be individual chips. Alternatively, the memory and the processor may be integrated in a single chip. The processor may be implemented as one or more processors. The processor may include various logic circuits and operation circuits, may be configured for processing data according to a program provided from the memory, and may be configured to generate a control signal according to the processing result.

The control device may be at least one microprocessor operated by a predetermined program which may include a series of commands for carrying out the method included in the aforementioned various exemplary embodiments of the present disclosure.

The aforementioned invention can also be embodied as computer readable codes on a computer readable recording medium. The computer readable recording medium is any data storage device that can store data which may be thereafter read by a computer system and store and execute program instructions which may be thereafter read by a computer system. Examples of the computer readable recording medium include Hard Disk Drive (HDD), solid state disk (SSD), silicon disk drive (SDD), read-only memory (ROM), random-access memory (RAM), CD-ROMs, magnetic tapes, floppy discs, optical data storage devices, etc and implementation as carrier waves (e.g., transmission over the Internet). Examples of the program instruction include machine language code such as those generated by a compiler, as well as high-level language code which may be executed by a computer using an interpreter or the like.

In various exemplary embodiments of the present disclosure, each operation described above may be performed by a control device, and the control device may be configured by a plurality of control devices, or an integrated single control device.

In various exemplary embodiments of the present disclosure, the memory and the processor may be provided as one chip, or provided as separate chips.

In various exemplary embodiments of the present disclosure, the scope of the present disclosure includes software or machine-executable commands (e.g., an operating system, an application, firmware, a program, etc.) for enabling operations according to the methods of various embodiments to be executed on an apparatus or a computer, a non-transitory computer-readable medium including such software or commands stored thereon and executable on the apparatus or the computer.

In various exemplary embodiments of the present disclosure, the control device may be implemented in a form of hardware or software, or may be implemented in a combination of hardware and software.

Furthermore, the terms such as “unit”, “module”, etc. included in the specification mean units for processing at least one function or operation, which may be implemented by hardware, software, or a combination thereof.

In an exemplary embodiment of the present disclosure, the vehicle may be referred to as being based on a concept including various means of transportation. In some cases, the vehicle may be interpreted as being based on a concept including not only various means of land transportation, such as cars, motorcycles, trucks, and buses, that drive on roads but also various means of transportation such as airplanes, drones, ships, etc.

For convenience in explanation and accurate definition in the appended claims, the terms “upper”, “lower”, “inner”, “outer”, “up”, “down”, “upwards”, “downwards”, “front”, “rear”, “back”, “inside”, “outside”, “inwardly”, “outwardly”, “interior”, “exterior”, “internal”, “external”, “forwards”, and “backwards” are used to describe features of the exemplary embodiments with reference to the positions of such features as displayed in the figures. It will be further understood that the term “connect” or its derivatives refer both to direct and indirect connection.

The term “and/or” may include a combination of a plurality of related listed items or any of a plurality of related listed items. For example, “A and/or B” includes all three cases such as “A”, “B”, and “A and B”.

In exemplary embodiments of the present disclosure, “at least one of A and B” may refer to “at least one of A or B” or “at least one of combinations of at least one of A and B”. Furthermore, “one or more of A and B” may refer to “one or more of A or B” or “one or more of combinations of one or more of A and B”.

In the present specification, unless stated otherwise, a singular expression includes a plural expression unless the context clearly indicates otherwise.

In the exemplary embodiment of the present disclosure, it should be understood that a term such as “include” or “have” is directed to designate that the features, numbers, steps, operations, elements, parts, or combinations thereof described in the specification are present, and does not preclude the possibility of addition or presence of one or more other features, numbers, steps, operations, elements, parts, or combinations thereof.

According to an exemplary embodiment of the present disclosure, components may be combined with each other to be implemented as one, or some components may be omitted.

The foregoing descriptions of specific exemplary embodiments of the present disclosure have been presented for purposes of illustration and description. They are not intended to be exhaustive or to limit the present disclosure to the precise forms disclosed, and obviously many modifications and variations are possible in light of the above teachings. The exemplary embodiments were chosen and described in order to explain certain principles of the invention and their practical application, to enable others skilled in the art to make and utilize various exemplary embodiments of the present disclosure, as well as various alternatives and modifications thereof. It is intended that the scope of the present disclosure be defined by the Claims appended hereto and their equivalents.

What is claimed is:

1. An apparatus for controlling a lower-limb wearable robot based on augmented reality and brain-computer interface, the apparatus comprising:
an augmented reality glasses configured to display a visual stimulus and status information of the lower-limb wearable robot through the augmented reality;
a biological signal measurement module configured for measuring a biological signal of a user generated based on the visual stimulus and the status information of the lower-limb wearable robot;
a control module configured to detect a control intention of the user based on the biological signal and generate the visual stimulus and the status information of the lower-limb wearable robot; and
the lower-limb wearable robot configured to operate according to the control intention of the user.

2. The apparatus of claim 1,
wherein the biological signal includes a first biological signal and a second biological signal, and
wherein the biological signal measurement module includes:
a first biological signal measurement module configured for measuring the first biological signal including electroencephalogram information of the user gazing at the visual stimulus; and
a second biological signal measurement module configured for measuring the second biological signal generated by the user based on the status information of the lower-limb wearable robot.

3. The apparatus of claim 2,
wherein the control intention of the user includes a first control intention and a second control intention, and
wherein the control module includes:
a brain-computer interface (BCI) calculating unit configured to classify the first control intention of the user based on the first biological signal and determine the second control intention of the user based on the second biological signal; and
an augmented reality (AR) calculating unit configured to generate the visual stimulus and the status information of the lower-limb wearable robot based on the second control intention.

4. The apparatus of claim 2, wherein the control module is further configured to:
divide the second biological signal into a first signal and a second signal;
switch a state of the lower-limb wearable robot from a stand state to a sit state based on the first signal in response that the state of the lower-limb wearable robot is a pause state; and
switch the state of the lower-limb wearable robot from the sit state to the stand state based on the second signal.

5. The apparatus of claim 2, wherein, in response that a state of the lower-limb wearable robot is a stand state in a pause state, the control module is further configured to switch the state of the lower-limb wearable robot to an operation state based on the second biological signal.

6. The apparatus of claim 5, wherein, in response that the state of the lower-limb wearable robot is the operation state, the control module is further configured to determine an operation mode based on the first biological signal.

7. The apparatus of claim 6, wherein, in response that the lower-limb wearable robot is in the operation mode, the control module is further configured to control an operation corresponding to the determined operation mode of the lower-limb wearable robot based on the second biological signal.

8. The apparatus of claim 7, wherein the control module is further configured to generate a crutch position guide configured to indicate a position to which a crutch of the user is to move, to correspond to the controlled operation of the lower-limb wearable robot in real time.

9. The apparatus of claim 8, wherein the augmented reality glasses is configured to display the crutch position guide at a second location spaced from a first location where the visual stimulus and the status information of the lower-limb wearable robot are displayed and closer to the ground than the first location.

10. The apparatus of claim 1, wherein the control module is further configured to generate a plurality of visual stimuli that are based on steady-state visual evoked potential (SSVEP) and flickering with different frequencies.

11. A method for controlling a lower-limb wearable robot based on augmented reality and brain-computer interface, the method comprising:
displaying visual stimulus and status information of the lower-limb wearable robot to a user through an augmented reality glasses;
measuring a biological signal of the user generated based on the visual stimulus and the status information of the lower-limb wearable robot;
generating, by a processor, the visual stimulus and the status information of the lower-limb wearable robot based on the biological signal;
detecting a control intention of the user based on the biological signal; and
controlling, by the processor, an operation of the lower-limb wearable robot according to the control intention of the user.

12. The method of claim 11,
wherein the biological signal includes a first biological signal and a second biological signal, and
wherein the measuring the user's biological signal includes:
measuring the first biological signal including electroencephalogram information of the user gazing at the visual stimulus; and
measuring the second biological signal generated by the user based on the status information of the lower-limb wearable robot.

13. The method of claim 12,
wherein the control intention of the user includes a first control intention and a second control intention, and
wherein the detecting the control intention of the user includes classifying the first control intention of the user based on the first biological signal, and determining the second control intention of the user based on the second biological signal; and
wherein the generating the status information of the lower-limb wearable robot includes generating the visual stimulus and the status information of the lower-limb wearable robot based on the second control intention.

14. The method of claim 12, wherein the generating the status information of the lower-limb wearable robot includes:
dividing the second biological signal into a first signal and a second signal;
switching a state of the lower-limb wearable robot from a stand state to a sit state based on the first signal, in response that the state of the lower-limb wearable robot is a pause state; and
switching the state of the lower-limb wearable robot from the sit state to the stand state based on the second signal.

15. The method of claim 12, wherein the generating the status information of the lower-limb wearable robot includes, in response that a state of the lower-limb wearable robot is a stand state in a pause state, switching the state of the lower-limb wearable robot to an operation state based on the second biological signal.

16. The method of claim 15, wherein the detecting the control intention of the user includes, in response that the state of the lower-limb wearable robot is the operation state, determining an operation mode based on the first biological signal.

17. The method of claim 16, wherein the controlling the operation of the lower-limb wearable robot further includes, in response that the lower-limb wearable robot is in the operation mode, controlling an operation corresponding to the determined operation mode of the lower-limb wearable robot based on the second biological signal.

18. The method of claim 17, further including generating a crutch position guide configured to indicate a position to which a crutch of the user is to move, to correspond to the controlled operation of the lower-limb wearable robot in real time.

19. The method of claim 18, further including displaying the crutch position guide at a second location spaced from a first location where the visual stimulus and the status information of the lower-limb wearable robot are displayed and closer to the ground than the first location through the augmented reality glasses.

20. The method of claim 11, wherein the generating the visual stimulus and the status information of the lower-limb wearable robot includes generating a plurality of visual stimuli that are based on steady-state visual evoked potential (SSVEP) and flickering with different frequencies.
